(19) United States
(12) Patent Application Publication
(10) Pub. No.: US 2011/0275090 A1
(43) Pub. Date: Nov. 10, 2011

(54) USE OF ACHROMOPEPTIDASE FOR LYSIS AT ROOM TEMPERATURE

(75) Inventors: Danielle Hilligoss, Westminster, MD (US); Lisa M. Keller, York, PA (US); Samah Ramadan, Hampstead, MD (US); Jamie Coady, Lutherville, MD (US); Tobin J. Hellyer, Westminster, MD (US)

(73) Assignee: BECTON, DICKINSON AND COMPANY, Franklin Lakes, NJ (US)

(21) Appl. No.: 13/048,539

(22) Filed: Mar. 15, 2011

Related U.S. Application Data
(60) Provisional application No. 61/314,318, filed on Mar. 16, 2010.

(51) Int. Cl. C12Q 1/68; C07H 1/08

(52) U.S. Cl. 435/6.15; 536/25.41

(57) ABSTRACT

A process for detecting the presence or absence of gram-positive bacteria in a biological sample. The biological sample can be obtained from any mammal and contains, at a minimum, cellular components. The sample is combined with an enzymatic lysing agent such as achromopeptidase, and lysed at room temperature. Ferric oxide is then added to the sample containing achromopeptidase. A magnetic field is applied to the sample and nucleic acids are extracted from the cellular components. Target nucleic acids, if present, are amplified using techniques such as Polymerase Chain Reaction (PCR) and then used to detect the presence or absence of gram-positive bacteria. Staphylococcus aureus and Streptococcus agalactiae are examples of target bacteria detected by the methods of the present invention.

### CROSS-REFERENCE TO RELATED APPLICATIONS

This application claims the benefit of the filing date of U.S. Provisional Patent Application No. 61/314,318 filed Mar. 16, 2010, the disclosure of which is hereby incorporated by reference.

### BACKGROUND OF THE INVENTION

Certain species of gram-positive bacteria are known pathogens. Staphylococcus aureus and Streptococcus agalactiae are two types of bacteria known to be the root cause of particularly virulent infections in mammals. Detection of these pathogens is critical for successful diagnosis and treatment.

There are many methods useful for the detection of pathogenic bacteria, several of which rely on lysis of the bacterial cell walls. After lysis, nucleic acids are subsequently extracted from the cellular components and amplified in downstream processes such as PCR. The presence or absence of nucleic acids are then used as an indicator of infection.

Lysis of gram-positive bacteria is particularly difficult, in part, due to the structure of their cell walls. Both gram-negative and gram-positive bacteria contain a peptidoglycan layer within their cell walls. This layer is comprised of glycan chains cross linked by peptide bridges. However, in gram-positive bacteria, the quantity, thickness and extent of cross-linking within the peptidoglycan layer is more extensive. Mahalanabis et al., Cell lysis and DNA extraction of gram-positive and gram negative bacteria from whole blood in a disposable microfluidic chip, Lab Chip, 9, 2811-17 (2009) This more robust peptidoglycan layer makes gram-positive bacteria challenging to lyse enzymatically.

The bacteriolytic enzyme achromopeptidase is an effective lysing agent of gram-positive bacteria. However, it is not without disadvantages. While achromopeptidase does effectively lyse the cell walls of gram positive bacteria, if its activity is not stopped after a certain period of time, it will continue to lyse other critical cellular constituents necessary for further downstream analysis, such as PCR.

Significantly increasing the sample temperature is one method of stopping the lytic activity of achromopeptidase. This increase in temperature, alternatively known as a heat spike, halts any lytic activity of the enzyme. However, creating a system whereby this type of heat is generated is expensive and adds significant complexity to the diagnostic platform. The present invention overcomes these challenges by eliminating the heat spike and using extraction methods to stop the lysing activity of achromopeptidase.

### BRIEF SUMMARY OF THE INVENTION

The present invention provides a method for extracting nucleic acids from a biological sample using achromopeptidase as a lysing agent. In one embodiment, the extraction begins with a biological sample. While the biological sample contains cellular components, it may also have other constituents as well. The biological sample is then combined with achromopeptidase and lysed at room temperature. As used herein, lysis is defined as the rupture of cell walls and cell membranes by external, mechanical or non-mechanical means. The method described herein achieves lysis without the use of mechanical means. In yet another embodiment, the sample is lysed at a specific temperature of between 18° C. and 22° C.

In a further embodiment, the sample is combined with achromopeptidase and 10% phosphate buffered saline (“PBS”) solution. The PBS Solution provides an isotonic environment for the biological sample, aids in maintaining the viability of the cellular components within the sample, and further provides a low salt environment with a controlled pH level. In addition to PBS solution, additional embodiments of the present invention utilize Amies Medium, Stuarts Medium and Tris EDTA as buffers.

After lysis, the sample is added to ferric oxide and the nucleic acids are extracted from the sample using a magnetic field. After extraction, the nucleic acids are then amplified and the presence or absence of gram positive bacteria in the sample is detected. Staphylococcus aureus and Streptococcus agalactiae are among the gram positive bacteria detected by the present invention. Additionally, the methods of the present invention are useful for targeting methicillin resistant Staphylococcus aureus (MRSA).

### BRIEF DESCRIPTION OF THE DRAWINGS

FIG. 1 illustrates one embodiment of the present invention that utilizes a sample swab having a tip that can be broken off and left within the sample tube;

FIG. 1A illustrates another embodiment of the present invention that utilizes a sample swab having a tip that can be broken off and left within the sample tube.

FIG. 2 is a perspective view of one embodiment of the present invention that utilizes a cylindrical sample tube with a tapered bottom capable of holding a biological sample;

FIG. 2A is a side view of another embodiment of the present invention that utilizes a cylindrical sample tube capable of holding a biological sample;

FIG. 2B is a bottom view of a cylindrical sample tube capable of holding a biological sample;

FIG. 2C is a cross section of FIG. 2 along A that utilizes a cylindrical sample tube with a tapered bottom capable of holding a biological sample;

FIG. 2D is a side view of another embodiment of the present invention that utilizes a cylindrical sample tube capable of holding a biological sample;

FIG. 3 is a perspective view of a third embodiment of the present invention that utilizes a piercable cap for sealing a sample tube;

FIG. 3A is a plan view of one embodiment of the present invention that utilizes a pierceable cap for sealing a sample tube;

FIG. 3B illustrates a side view of one embodiment of the present invention that utilizes a pierceable cap for sealing a sample tube;

FIG. 3C illustrates a cross section of FIG. 3 along B that utilizes a pierceable cap for sealing a sample tube containing two sealing membranes.

### DETAILED DESCRIPTION

Described herein is a method for lysing cells using an enzyme with lytic properties. Any type of cell may be lysed by the methods discussed. In preferred embodiments, gram positive bacterial cells are lysed. However, both gram positive and gram negative bacteria may be lysed using the methods of the present invention. The method contemplates extracting nucleic acid from the lysed bacteria. The extracted nucleic acid is then used for purposes known to those skilled in the art (i.e. diagnosis and detection of the target from which the nucleic acid is extracted). Since the uses of nucleic acids for purposes of diagnosis and detection is well known, assays and the like for the isolation and detection of target nucleic acid are not described in detail herein. In a preferred embodiment, the target nucleic acid is DNA.

Any bacterial cell may be lysed using the methods described herein. Therefore, the present invention is useful for the diagnosis and detection of a wide array of bacterial species. Examples of bacterial species useful in the present invention include, but are not limited to, methicillin resistant Staphylococcus aureus (MRSA) and Streptococcus agalactiae (GBS). Other species of bacteria that can be detected by the method described herein are Chlamydia trachomatis and Neisseria gonorrhoeae.

Samples that are tested for the presence or absence of target bacteria according to the method described herein are collected using any conventional method, and are in no way limited to the source of the sample. Any potential source of bacteria can be analyzed by the methods described herein.

Sample collection can be through conventional means such as a swab, blood draw, urine sample, etc. In one preferred embodiment, samples can be obtained from a vagino-rectal swab. In another preferred embodiment, the bacterial samples can be obtained from nasal swabs. Samples can be collected from urine, semen, sputum, blood, saliva, mucus, feces or any other tissue or fluid derived from a human or animal using any of the aforesaid conventional methods of collection.

In other embodiments of the present invention, samples can be collected from the environment, including but not limited to water and soil samples. Such samples are collected using the conventional methods noted above, such as swabs. In addition to water and soil, any surface or object can be swabbed by those methods known in the art and analyzed by the methods described herein. The inventors fully contemplate using embodiments of the present invention to detect the presence of bacteria at various locations throughout the environment. This includes but is by no means limited to the surfaces of objects such as counters, door knobs, car handles, bathroom surfaces and any other physical location within the environment that one skilled in the art may believe to contain bacteria.

In the described method, achromopeptidase is used to lyse the bacteria to release the nucleic acid from the organism for extraction from the sample for detection. The nucleic acid is the signature indication of the presence of the target bacteria in the sample. As achromopeptidase is known to be sensitive to increased salinity, certain steps, described in detail below, are preferably taken to obtain and prepare samples for lysis using achromopeptidase.

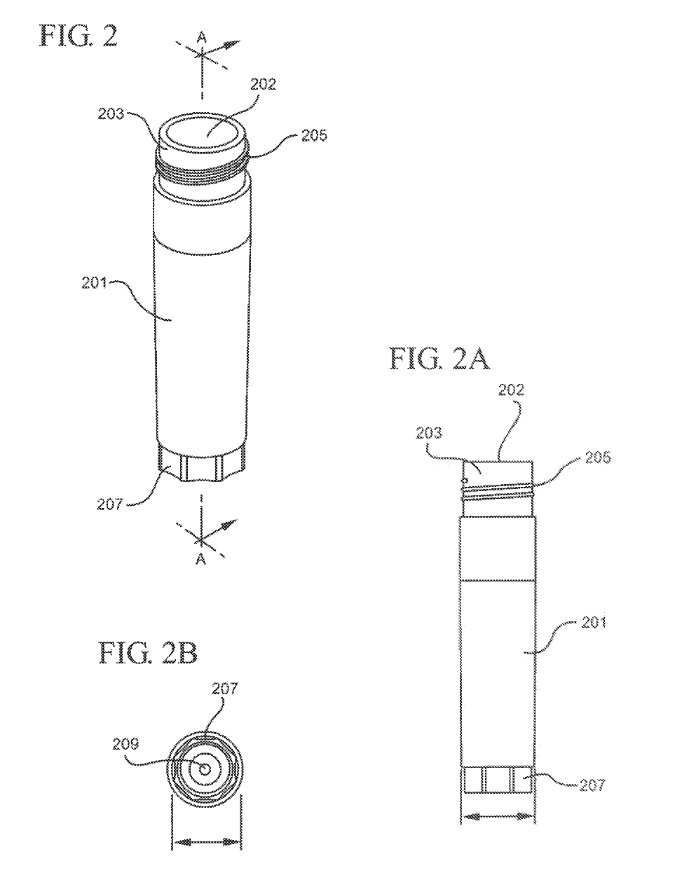

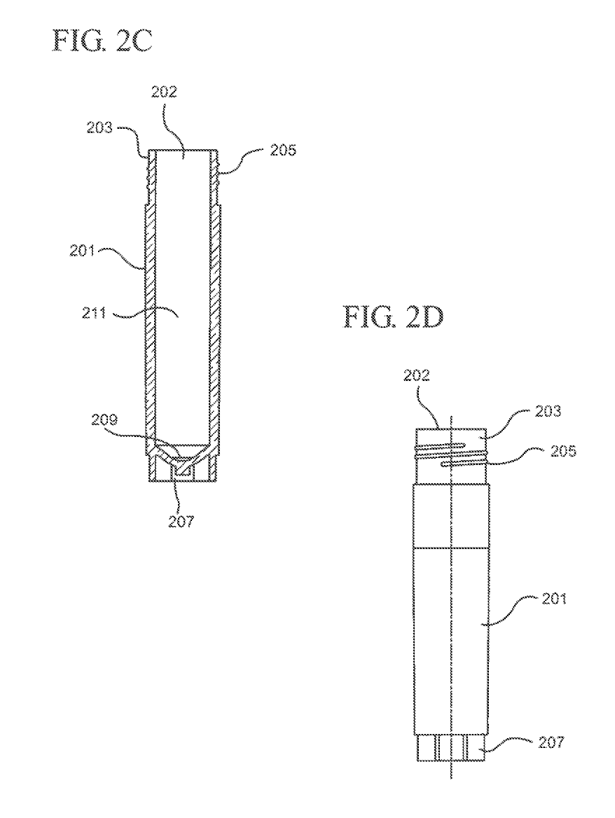

In one embodiment, specimens are collected via a collection and transport device. One example of a transport device is illustrated in FIG. 2. The transport device is a system wherein a wet swab is used to collect a sample that may contain target organisms, such as MRSA, GBS, etc.

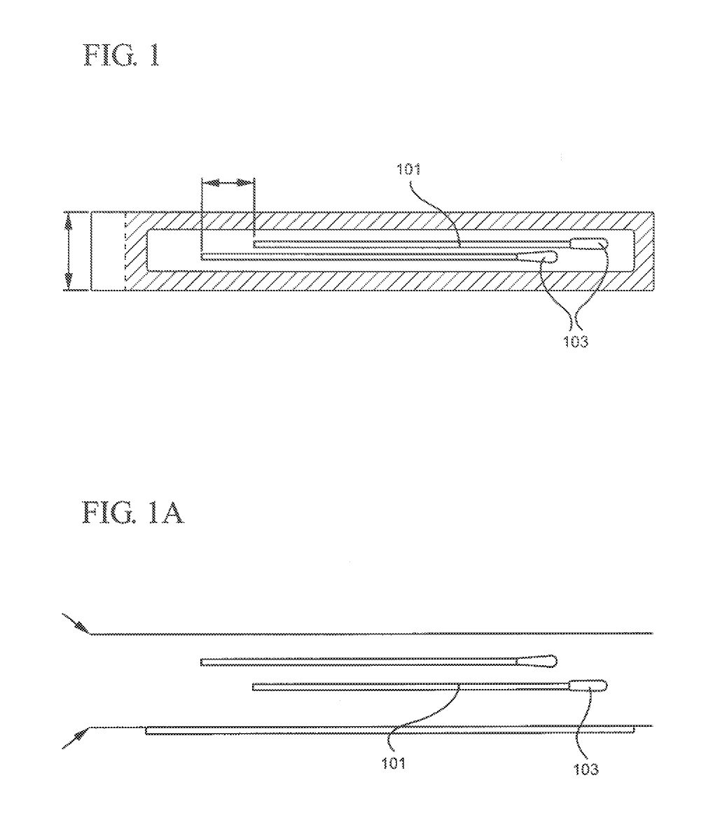

One embodiment of the present invention uses the swab illustrated in FIGS. 1 and 1A. The purpose of the wet swab collection device is to facilitate onsite sample collection and prolong viability of the collected organisms. The swab itself has a tip 103 configured for obtaining the swab sample. The tip 103 may be designed by means commonly known to those of ordinary skill in the art. The swab may also contain a perforation 101, so that after swabbing a sample site, e.g. a swab of the nasal passage, the collection portion may be broken off into the transport tube 201 at the collection site.

The transport tube 201 is illustrated in FIGS. 2, 2A, 2B, 2C and 2D. The tube 201 may be generally cylindrical in shape and contain an opening 202, and a sample space 211. The tube 201 contains a top portion 203 which may have threads disposed thereon, and a bottom portion 207. In one embodiment, the bottom portion 207 may be tapered 209.

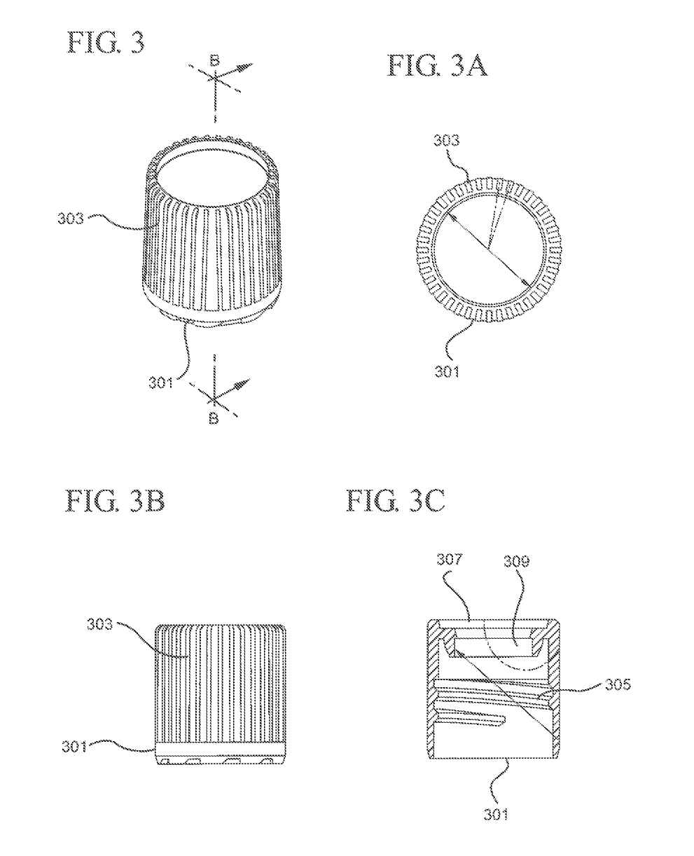

The transport tube 201 in which the swab is held may be adapted to receive a pierceable cap 301, as illustrated in FIGS. 3, 3A, 3B, and 3C. After collection, the swab is broken into the tube 201 and the tube 201 is sealed by the pierceable cap.

In one preferred embodiment, the pierceable cap 301 is configured with raised protrusions 303 disposed in an axial direction, for facilitating a manual grip on the cap 301. The cap 301 may also be threaded (305) to securely attach the cap 301 to the threads 205 of the tube 201. In a preferred embodiment, the cap 301 may contain an upper membrane 307 and lower membrane 309 for reducing aerisolization and contamination. Incorporated by reference herein are commonly assigned U.S. patent Ser. Nos. 11/785,144 and 11,979,713, which describe commonly assigned structures and designs of pierceable caps and their methods of use and that are not described in detail herein.

Preferably, the transport medium contained within the tube is selected to preserve viability for potential future culture. In one preferred embodiment, the transport medium is 10% Phosphate Buffered Saline (“PBS”) solution. As discussed herein, this solution aids in maintaining the viability of the cellular components, and also provides a low salt environment with the controlled pH that is advantageous for achromopeptidase lysis. Because samples are extracted from the transport medium for lysis and detection, it is important that the transport medium, which is extracted along with the samples, does not impede or otherwise adversely affect lysis.

In particular, transport mediums with properties similar to those of human body fluids are useful in preserving cellular integrity and organism viability. In this regard the use of 10% PBS solution is preferred, and the transport device discussed herein is not tied to the use of any particular transport medium. Consequently, any conventional transport medium is contemplated as suitable for use with the present invention, so long as it meets the criteria of preserving cell viability as previously described. Such transport media are well known to the skilled person and not described in detail herein. Other transport mediums may include, but are not limited to Stuarts Medium, Amies Medium, and Tris EDTA (“TE”). Dilution of these media in order to create an environment with the above-described salinity and pH environment is preferred. As discussed herein, media that create an isotonic environment may provide an optimal lysing environment.

As noted above, high saline content inhibits the lysing activity of achromopeptidase. Certain low saline mediums, such as those described above, preserve the activity of achromopeptidase. In one preferred embodiment, the transport medium is the solution of 10% PBS solution described above. This dilute solution is prepared using a 100% PBS solution that is prepared by mixing 0.023 mM Monobasic Potassium Phosphate, 0.629 mM Dibasic Potassium Phosphate, 14.5 mM Sodium Chloride and water. In another embodiment a 10% to 50% Stuarts medium is used. Table 1 illustrates the constituents in Stuarts Medium.

TABLE 1
100% Stuarts Medium

| Reagent | Amount |
|---|---|
| Calcium Chloride | 0.10 g |
| Sodium Chloride | 1 mL |
| Sodium Glycerophosphate | 10 g |

In yet another preferred embodiment, 10% to 50% Amies medium can be used. Table 2 depicts the composition of 100% Amies medium.

TABLE 2
100% Amies Medium

| Reagent | Amount |
|---|---|
| Calcium Chloride | 0.1 g/L |
| Disodium Phosphate | 1.15 g/L |
| Magnesium Chloride | 0.2 g/L |
| Monopotassium Phosphate | 0.2 g/L |
| Potassium Chloride | 0.2 g/L |
| Sodium Chloride | 0.2 g/L |
| Sodium Thioglycolate | 1.0 g/L |

In part, PBS, Amies Medium, Stuarts Medium, and TE are useful buffers in conjunction with enzymatic lysis, and in particular achromopeptidase lysis, because they provide an isotonic environment similar to that found in human body fluids.

In yet another preferred embodiment, the collection tube with the sample and medium described above is configured such that it can be placed directly into a device that assays the sample for the presence of target nucleic acid or any further analysis. This underscores the need for synergy between the transport medium and the lysis environment. After the organisms in the sample within the collection tube are lysed, the tube can be placed directly into a tool for the automated extraction and assay for the presence or absence of target nucleic acid. One such tool is the Viper™XTR platform which is commercially available from (Becton Dickinson, Sparks, Md.).

One embodiment described herein provides for the enzymatic lysis of a bacterial sample, extraction of the DNA from the sample and the subsequent use of the extracted bacterial DNA in diagnostic procedures. Also as noted above, preferably achromopeptidase (“ACH”), also known as lysyl endopeptidase, is used as a bacteriolytic enzyme to lyse the bacteria Staphylococcus aureus and Streptococcus agalactiae. Achromopeptidase possesses bacteriolytic, as well as proteolytic properties. While achromopeptidase is useful as a general bacteriolytic agent, it is particularly useful for lysing gram positive organisms, which are resistant to other bacteriolytic enzymes e.g. lysozyme. This resistance is thought to be linked to chemicals present in the cell walls of gram positive bacteria, but not present in gram negative bacteria. That being said, the method described herein is not limited to lysis with achromopeptidase, and can be practiced using any enzymatic lysing agent e.g. lysozyme.

Achromopeptidase is known to be an effective lytic enzyme when incubated with a bacterial sample, at temperatures ranging from 37° C. to 50° C. However, lysozymes such as achromopeptidase must be inactivated post lysis, because their continued proteolytic activity adversely affects subsequent diagnosis and detection of extracted nucleic acids. Means to achieve the cessation of lytic activity include performing a heat spike on the sample to stop the proteolytic action of the achromopeptidase. For purposes of the present invention, “heat spike” is defined as an increase in the sample temperature to about 95° C. One conventional approach to providing a heat spike is to heat the block on which the sample is placed to 95° C. for five minutes.

Once the sample is lysed, the nucleic acid can be extracted from the remainder of the sample components. In the methods described herein, any mechanism of nucleic acid extraction known in the art is contemplated as useful. Such mechanisms are well known and not described in detail herein. In one preferred embodiment, extraction is performed on the Viper™XTR. The Viper™XTR combines non-selective nucleic acid extraction using FOX particles from an extraction solution that contains KOH and other constituents.

The interplay between lysis, extraction solutions, and physical conditions are complicated and interdependent. In these environments it is difficult to draw the line precisely between where lysis ends and extraction begins. Also, the effects of the lysis solution on the extraction mechanisms are not well understood. That being said, disclosed herein is a particularly advantageous combination of lysis and extraction conditions that permit a room temperature lysis using achromopeptidase. This combination obtains the full benefits of achromopeptidase for lysis and avoids the negative effects of achromopeptidase on extracted nucleic acid. While the applicants do not wish to be held to a particular theory, applicants believe that the room temperature lysis using achrompeptidase, followed by non-selective nucleic extraction using ferric oxide particles in KOH and other extraction solution constituents used therewith, provides a particularly advantageous lysis and extraction protocol. This preferred embodiment does not preclude the use of other extraction protocols along with the room temperature achromopeptidase lysis described herein, so long as the negative effect of achromopeptidase on extracted nucleic acid is avoided. An investigation into the selection of alternative extraction protocols is well within the abilities of the skilled person.

In one aspect of the method described herein, incubation with achromopeptidase, followed by nucleic acid extraction with ferric oxide (Fe₂O₃)(“FOX”) particles is performed at room temperature. During the incubation and extraction, both the sample temperature and ambient temperature are room temperature. As used herein, “room temperature” is defined as a temperature in the range of about 16° C. to about 22° C. Alternatively, lysis with achromopeptidase can be conducted at temperatures higher than room temperature. However, lysis at these higher temperatures is less preferred.

Increasing the concentration of achromopeptidase in a sample allows lysis to occur at lower temperatures. However, higher concentrations of achromopeptidase may make analysis more difficult and may require longer incubation times. According to the method described herein, incubation and extraction at room temperature, without a heat spike, permits the use of a greater concentration of achromopeptidase without the attendant difficulties previously encountered. The result is a shorter incubation time and potentially greater usable yield of nucleic acid.

Following lysis of the cells in the sample, the nucleic acid is extracted from the rest of the lysed sample. Extraction is performed by introducing ferric oxide (Fe₂O₃) (“FOX”) particles to the bacterial sample. The FOX particles bind to the negatively charged DNA of the lysed sample. Magnets are then applied to the sample to attract the bound DNA and the eluent is removed by conventional means. This extraction procedure is successful in extracting DNA from lysed bacteria. FOX particles are used for nucleic acid extraction in the BD Viper™ System.

Once the DNA is extracted, further analysis can be performed for purposes of diagnosis and detection. Examples of downstream analysis include, but are not limited to, polymerase chain reaction, gel electrophoresis, etc. Platforms for biological testing biological samples for the presence or absence of target nucleic acid extraction from the samples include, the BD Viper™ System.

Protocol for Examples

In the following examples, conditions needed to lyse and subsequently extract DNA from bacteria were investigated. The two bacterial organisms present in all of the following examples were, Methicillin resistant Staphylococcus aureus (“MRSA”), ATCC #43300, subspecies aureus Rosenbach, and Streptococcus agalactiae (“GBS”), ATCC #12973, designated by ATCC as typing strain V8. The lysozyme i.e. achromopeptidase (“ACH”) (obtained from Wako Chemicals USA) was used as the lysing agent in all of the following examples.

To demonstrate the lytic effects of ACH on MRSA and GBS (collectively, “bacterial samples”), the bacterial samples were combined with varying concentrations of achromopeptidase and incubated at room temperature for varying times.

For each of the following examples, 7.5×10⁴ CFU/ml of organism was combined with the other constituents described in each example. The effect of the constituents and conditions on lysis and nucleic acid extraction was examined.

Nucleic acid extraction was performed utilizing iron oxide (FOX) technology on the BD Viper™ System. The BD Viper™ System is commercially available and its operation is not described in detail herein. First, lysed bacterial samples were placed into the BD Viper™ System. The bacterial samples were combined with FOX particles which bind to the nucleotide fragments of the lysed bacterial nucleic acids, including the bacterial DNA. Next, the samples were subjected to a magnetic field to isolate the bound nucleic acid from the other portions of sample. After isolation with the magnetic field, the other components of the sample were removed. The nucleic acid components were then eluted from the FOX particles in preparation for PCR. No heat spike was employed during the incubation process to stop the lysing action of ACH.

The result of the lysis procedures and subsequent DNA extraction was measured by the cycle threshold (“Ct”). As used herein, cycle threshold is defined as the fractional cycle number at which fluorescence passes a fixed threshold. Cycle threshold is a well known technique for determining a positive indication of a clinically significant amount of nucleic acid in a sample and is not described in detail herein.

For MRSA, a cycle consisted of increasing the temperature of the extracted DNA sample to 95° C. for 15 seconds, followed by a 59° C. exposure for 60 seconds. For GBS, a cycle consisted of increasing the temperature of the extracted DNA sample to 95° C. for 15 seconds, followed by a 56° C. exposure for 60 seconds. Forty five cycles were run for MRSA and GBS respectively. The cycle number corresponding to the fluorescent reading that exceeds a cycle threshold is the Ct.

A Ct value below 30 indicated an abundant amount of target nucleic acid in a sample. For purposes of the examples described herein, Ct values between 30-35 represented a moderate to low positive reaction and was considered a good result. Ct values between 35 and 45 represented weak reactions and indicate that only a minimal amount of DNA was extracted from the sample. A Ct value above 45 represented a sample wherein no DNA could be detected.

### EXAMPLE 1

### Room Temperature Lysis

MRSA and GBS bacteria were grown in cultures and then diluted in individual test tubes, each containing one mL solution of 1× TE (10 mM Tris/1 mM EDTA) to obtain a bacterial concentration in solution of about 7500 CFU/mL. Consequently the samples were spiked to contain target organisms.

ACH was dissolved in 1× TE and combined with the diluted bacterial samples. The concentration of ACH in each sample ranged from 1.01 U/μl to 5.05 U/μL. The resulting suspension of bacteria and achromopeptidase was incubated at either 22° C. or 37° C. for a range of 10 minutes to 30 minutes. The final concentrations of ACH in each one mL sample of bacteria ranged from about 1000 U to about 5000 U.

Following incubation, nucleic acid, including genomic DNA, was extracted using the BD Viper™ System according to the protocol described herein. After extraction, PCR was performed to amplify the extracted DNA.

Immediately after extraction, PCR was performed on the ABI 7500 Sequence Detection System (Applied Biosystems). A 50 uL PCR reaction was set up for each sample containing the following components: 200 uM of dNTPs (deoxyribonucleotide triphosphate); 2 U of FastStart Taq polymerase; 0.9 uM of right and left primer (Routing 04738403001); 0.25 uM of target specific molecular beacon; and 60 nM of ROX (reference dye), all in a commercially available PCR buffer (Roche)].

The thermal profile used during PCR in the samples containing MRSA and GBS was: 50° C. for 2 minutes; 95° C. for 10 minutes; and 45 cycles at 95° C. for 15 seconds and 59° C. for 1 minute.

Following amplification, all samples tested had Ct values less than 35.0. These Ct values indicated that MRSA and GBS DNA could be extracted with FOX technology, following room temperature lysis with ACH, without implementing a heat spike to stop lysis.

### EXAMPLE 2

### Room Temperature Lysis with Broader Range of ACH Concentration

Bacterial samples were prepared according to the protocol described herein and diluted using a solution of 1× TE to obtain a concentration of about 7500 CFU/mL.

The sample containing diluted bacteria was combined with a solution of achromopeptidase dissolved in 1× TE, ranging in concentration from 3.03 U/uL to 7.07 U/uL of ACH for final concentrations of 3000 U to 7000 U. The samples containing bacteria and achromopeptidase were incubated at room temperature for 20 minutes to allow for lysis.

Following incubation, nucleic acid, including genomic DNA, was extracted using the BD Viper™ System according to the protocol described herein. After extraction, PCR was performed to amplify the extracted DNA.

PCR was performed on the ABI 7500 Sequence Detection System (Applied Biosystems). A 50 uL PCR reaction was set up for each sample [200 uM dNTPs, 2 U FastStart Taq polymerase, 0.9 uM right and left primer, 0.25 uM target specific molecular beacon, 60 nM ROX, all in a commercially available PCR buffer (Roche)]. The following thermal profiles used for MRSA were: 50° C. for 2 minutes; 95° C. for 10 minutes; 45 cycles, at 95° C. for 15 seconds, 59° C. for 1 minute. The following thermal profiles used for GBS: 50° C. for 2 minutes; 95° C. for 10 minutes; 45 cycles at 95° C. 15 seconds and 56° C. for 1 minute.

Best results were obtained from samples lysed with 4000 to 6000 U of ACH. All of these samples gave Ct values of less than 35.0, indicating that a room temperature lysis utilizing a broad range of ACH concentrations was feasible when combined with FOX extraction technology.

### EXAMPLE 3

### Room Temperature Lysis with Two Levels of ACH Concentration and Titration of Organism Levels

MRSA and GBS samples were prepared by a growth culture and then diluting the samples to the five different testing levels. The samples were diluted in 1× TE to the following levels: 75000, 35000, 7500, 5000 and 1000 CFU/mL. Achromopeptidase was dissolved in 1× TE. The diluted bacterial samples were combined with a solution of dissolved achromopeptidase at a concentration of either 3.03 or 5.05 U/uL for final ACH concentrations of 3000 U or 5000 U. Bacterial samples and achromopeptidase were incubated at 22° C. for 20 min.

Following incubation, nucleic acid, including genomic DNA was extracted using the BD Viper™ System according to the protocol described herein. After extraction, PCR was performed to amplify the extracted DNA.

PCR was performed on the ABI 7500 Sequence Detection System (Applied Biosystems). A 50 uL PCR reaction was set up for each sample [200 uM dNTPs, 2 U FastStart Taq polymerase, 0.9 uM right and left primer, 0.25 uM target specific molecular beacon, 60 nM ROX, all in a commercially available PCR buffer (Roche)].

The following thermal profiles used for MRSA were: 50° C. for 2 minutes; 95° C. for 10 minutes; 45 cycles, at 95° C. for 15 seconds, 59° C. for 1 minute. The following thermal profiles used for GBS: 50° C. for 2 minutes; 95° C. for 10 minutes; 45 cycles at 95° C. 15 seconds and 56° C. for 1 minute.

All samples at all target levels were detected and Ct values ranged from 27.5 to 36.2. Results did not show a practical difference between either concentration of ACH tested. The Ct values indicate that a room temperature lysis, utilizing multiple ACH concentrations was feasible when combined with FOX extraction technology, and yielded an amount of DNA sufficient to perform analyses known to those in the art e.g. PCR.

### EXAMPLE 4

### Ambient Temperature Extraction Data

Testing for GBS and MRSA organisms in the TE buffer, was performed according to the method described in Example 2. The organisms in this experiment were lysed using achromopeptidase at 37° C. for 30 minutes. A subset of the experimental subjects underwent a heat kill at 95° C. for 5 minutes, followed by extraction with FeO. Another subset underwent FeO extraction without a heat kill, and a final subset of organisms went directly into a PCR reaction without any extraction (but both with and without heat kill). As a control, another set of samples were not lysed and not subjected to a heat kill, but were subjected to extraction and analysis. Tables 3 and 4 below indicate the results for GBS and MRSA, respectively. As indicated by Tables 3 and 4, each subset underwent two repetitions. This is indicated by the designations “rep 1” and “rep 2”. Next, for each repetition, two PCRs were performed. The cycle thresholds (“Ct”) are indicated in Tables 3 and 4. For all samples, a lower Ct indicated a higher recovery of DNA from the original sample. For the samples that did not undergo extraction, PCRs were performed directly on each sample after lysis.

TABLE 3
GBS-Extracted Samples

| | Lysed @ 37 C., 30′ | | | | No Lysis | | | |
|---|---|---|---|---|---|---|---|---|
| | Heat kill @ 95 | | No Heat Kill | | No Heat Kill | | | |
| | rep 1 | rep 2 | rep 1 | rep 2 | rep 1 | rep 2 | gDNA | PC |
| cfu/rxn | 3000 | 3000 | 3000 | 3000 | 3000 | 3000 | 3000 | 1000 |
| Ct | 28.85 | 30.76 | 29.11 | 30.98 | 39.81 | 39.69 | 37.30 | 31.54 |
| | 29.36 | 29.40 | 30.98 | 30.78 | 43.77 | 38.96 | 35.62 | 31.92 |
| Mean | 29.10 | 30.08 | 30.04 | 30.88 | 41.79 | 39.32 | 36.46 | 31.73 |
| Sdev | 0.36 | 0.96 | 1.32 | 0.15 | 2.80 | 0.52 | 1.19 | 0.27 |

Direct to PCR (3 ul)

| | Lysed @ 37 C., 30′ | | | | |
|---|---|---|---|---|---|
| | Heat kill @ 95 | | No Heat Kill | | |
| | rep 1 | rep 2 | rep 1 | rep 2 | gDNA |
| cfu/rxn | 225 | 225 | 225 | 225 | 225 |
| Ct | 31.06 | 30.65 | 40.72 | 43.43 | 33.81 |
| | 30.63 | 30.67 | 44.72 | U | 33.50 |
| Mean | 30.85 | 30.66 | 42.72 | 43.43 | 33.65 |
| Sdev | 0.31 | 0.02 | 2.83 | U | 0.22 |

TABLE 4
MRSA-Extracted Samples

| | Lysed @ 37 C., 30′ | | | | No Lysis | | | |
|---|---|---|---|---|---|---|---|---|
| | Heat kill @ 95 | | No Heat Kill | | No Heat Kill | | | |
| | rep 1 | rep 2 | rep 1 | rep 2 | rep 1 | rep 2 | gDNA | PC |
| cfu/rxn | 3000 | 3000 | 3000 | 3000 | 3000 | 3000 | 3000 | 1000 |
| Ct | 30.05 | 28.73 | 30.35 | 31.25 | 35.40 | 34.97 | 35.56 | 32.41 |
| | 29.94 | 28.34 | 30.78 | 32.63 | 34.70 | 34.74 | 35.89 | 31.57 |
| Mean | 30.00 | 28.54 | 30.57 | 31.94 | 35.05 | 34.85 | 35.72 | 31.99 |
| Sdev | 0.08 | 0.28 | 0.30 | 0.97 | 0.50 | 0.16 | 0.23 | 0.59 |

Direct to PCR (3 ul)

| | Lysed @ 37 C., 30′ | | | | |
|---|---|---|---|---|---|
| | Heat kill @ 95 | | No Heat Kill | | |
| | rep 1 | rep 2 | rep 1 | rep 2 | gDNA |
| cfu/rxn | 225 | 225 | 225 | 225 | 225 |
| Ct | 30.02 | 27.58 | 36.41 | 38.06 | 33.48 |
| | 30.52 | 30.23 | U | 33.36 | 34.32 |
| Mean | 30.27 | 28.90 | 36.41 | 35.71 | 33.90 |
| Sdev | 0.36 | 1.87 | U | 3.32 | 0.59 |

As indicated by Tables 3 and 4, organisms lysed at 37° C. for 30 minutes and extracted without a heat kill had similar results to those organisms which underwent a heat kill. Any mean Ct count below 35 was considered a positive recovery. As indicated by both Table 3 and Table 4, organisms not lysed and not subjected to a heat kill had Ct counts greater than 39 for GBS and greater than 34.5 for MRSA. Organisms not lysed and not subjected to a heat kill were used to show that the experimental groups were in fact effective at extracting DNA from the sample organisms.

In the cases of both GBS and MRSA, the mean Ct counts for lysed organisms that were not exposed to a heat kill were essentially equivalent to those lysed organisms that were exposed to a heat kill. These results indicate that a clinically useful amount of DNA can be successfully extracted from bacteria, with achromopeptidase, and extracted with FeO without a heat kill.

Although the invention herein has been described with reference to particular embodiments, it is to be understood that these embodiments are merely illustrative of the principles and applications of the present invention. It is therefore to be understood that numerous modifications may be made to the illustrative embodiments and that other arrangements may be devised without departing from the spirit and scope of the present invention as defined by the appended claims.

1. A method for extracting nucleic acids from target bacteria if present in a sample comprising:
a) lysing at room temperature, a biological sample comprising cellular components, wherein the biological sample is combined with achromopeptidase;
b) combining ferric oxide particles with the biological sample wherein nucleic acid from the lysed sample binds to the ferric oxide; and
c) magnetically separating nucleic acids bound to the ferric oxide particles from the biological sample.

2. The method of claim 1, wherein the nucleic acids are extracted from the biological sample using a magnetic field.

3. The method of claim 2, wherein the ferric oxide particles are uncoated.

4. The method of claim 1, wherein the biological sample is combined with a buffer.

5. The method of claim 4, wherein the buffer creates an isotonic environment for the biological sample.

6. The method of claim 4, wherein the buffer maintains the viability of the cellular components.

7. The method of claim 4, wherein the buffer creates a low salt environment with controlled pH.

8. The method of claim 5, wherein the buffer is selected from the group consisting of:10% Phosphate Buffered Saline Solution; Amies Medium; Stuarts Medium and Tris EDTA.

9. The method of claim 1, wherein the target bacteria is gram positive bacteria.

10. The method of claim 9, wherein the target gram positive bacteria is selected from the group consisting of Staphylococcus aureus and Streptococcus agalactiae.

11. The method of claim 1, wherein the biological sample is collected from a group consisting essentially of urine, semen, sputum, blood, saliva, mucus and feces.

12. The method of claim 1, wherein the biological sample is collected from the environment.

13. The method of claim 12, wherein the biological sample is collected from water or soil.

14. The method of claim 12, wherein the sample is collected from any object surface found in the environment.

15. A method for extracting nucleic acids of target gram positive microorganisms if present in a biological sample comprising:
a) lysing at room temperature, a biological sample comprising cellular components, wherein the biological sample is combined with achromopeptidase and 10% Phosphate Buffered Saline Solution;
b) combining ferric oxide particles with the biological sample wherein nucleic acid, if present from the lysed biological sample, binds to the ferric oxide particles; and
c) magnetically separating nucleic acids bound to the ferric oxide particles from the biological sample.

16. The method of claim 15, wherein the target gram positive microorganism is methicillin resistant Staphylococcus aureus (MRSA).

17. The method of claim 15, wherein room temperature is in the range of about 18° C. to about 22° C.

18. A method for detecting the presence or absence of target microorganisms if present in a biological sample comprising:
a) combining a biological sample with achromopeptidase wherein the biological sample comprises at least a cellular component;
b) lysing at least a portion of the cellular component of the biological sample at a temperature of about 18° C. to about 22° C.;
c) combining ferric oxide particles with the biological sample;
d) binding nucleic acids, if present, from the lysed cellular components to at least some of the ferric oxide particles;
e) isolating nucleic acids from the biological sample using a magnetic field including, if present, nucleic acids from the cellular components of the target microorganisms;
f) amplifying the isolated nucleic acids from the cellular components of the target microorganisms, if present;
g) conducting an assay for detecting the presence or absence of nucleic acid of the target microorganisms; and
h) determining presence or absence of target microorganisms based on the detected presence or absence of nucleic acid of the target microorganisms in the assay.

19. The method of claim 18, wherein the target microorganisms are gram positive bacteria.

20. The method of claim 19, wherein the gram positive bacteria is selected from the group consisting of Staphylococcus aureus and Streptococcus agalactiae.

21. The method of claim 18 wherein the biological sample is combined with a buffer selected from the group consisting of 10% Phosphate Buffered Saline Solution, Amies Medium, Stuarts Medium and Tris EDTA.

22. The method of claim 18 wherein the biological sample is maintained at a temperature of about 18° C. to about 22° C. for at least steps b) through e).
